(19) United States
(12) Patent Application Publication
(10) Pub. No.: US 2011/0092602 A1
(43) Pub. Date: Apr. 21, 2011

(54) RAPIDLY DISINTEGRATING ORAL COMPOSITIONS OF TRAMADOL

(76) Inventors: Nitin Shivcharan Laddha, Gujarat (IN); Sushrut K. Kulkarni, Gujarat (IN); Sunilendu Bushan Roy, Gujarat (IN)

(21) Appl. No.: 12/937,861

(22) PCT Filed: Apr. 24, 2009
(86) PCT No.: PCT/IN09/00251

§ 371 Date: Dec. 29, 2010

(30) Foreign Application Priority Data
Apr. 25, 2008 (IN) 915/MUM/2008

(51) Int. Cl. A61K 31/135

(52) U.S. Cl. 514/646

(57) ABSTRACT

The present invention relates to a stable taste masked orally disintegrating pharmaceutical composition of tramadol comprising of 3% to 30% w/w of tramadol, 2% to 30% w/w of an ion exchange resin, 0.01 to about 2% w/w of binder and pharmaceutically acceptable excipients to equal 100% w/w, wherein the binder used is gelatin.

### FIELD OF INVENTION

The present invention relates to orally disintegrating pharmaceutical composition of Tramadol or its pharmaceutically acceptable salts or solvates.

The present invention relates to compositions for Tramadol in the form of oral composition and process for obtaining them. Tramadol is currently marketed as a hydrochloride salt, which is the generic name for the compound of formula (I) (±)cis-2-[(dimethylamino)methyl]-1-(3-methoxyphenyl)cyclohexanol hydrochloride.

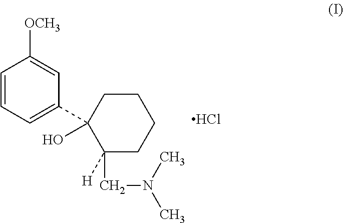

### BACKGROUND OF THE INVENTION

Tramadol hydrochloride is a white, bitter, crystalline and odorless powder. It is readily soluble in water and ethanol and has a pKa of 9.41. The n-octanol/water log partition coefficient (logP) is 1.35 at pH 7. The molecular weight of tramadol hydrochloride is 299.8.

Tramadol hydrochloride is (±)cis-2-[(dimethylamino)methyl]-1-(3-methoxyphenyl)cyclohexanol. It is a atypical opioid which is a centrally acting analgesic, used for treating moderate to severe pain. It is a synthetic agent, as a 4-phenyl-piperidine analogue of codeine, and appears to have actions on the GABAergic, noradrenergic and serotonergic systems. Tramadol was developed by the German pharmaceutical company Grünenthal GmbH in the last years of 1970s and marketed under the trade name Tramal®. Grünenthal has also cross licensed the drug to many other pharmaceutical companies that market it under various names.

Tramadol is usually marketed as the hydrochloride salt (tramadol hydrochloride) and is available in both injectable (intravenous and/or intramuscular) and oral preparations. It is also available in conjunction with paracetamol (acetaminophen).

Tramadol is marketed in the USA under the trademark ULTRAM®. ULTRAM® tablets are available as 50 mg white tablets for oral administration. Tramadol hydrochloride in the form of orally disintegrating tablets has been approved by the USFDA.

Tramadol is a centrally acting synthetic opioid analgesic. Although its mode of action is not completely understood, from animal tests, at least two complementary mechanisms appear applicable: binding of parent and M1 metabolite to μ-opioid receptors and weak inhibition of reuptake of norepinephrine and serotonin.

Opioid activity is due to both low affinity binding of the parent compound and higher affinity binding of the O-demethylated metabolite M1 to μ-opioid receptors. In animal models, M1 is up to 6 times more potent than tramadol in producing analgesia and 200 times more potent in μ-opioid binding. Tramadol-induced analgesia is only partially antagonized by the opiate antagonist naloxone in several animals tests. The relative contribution of both tramadol and M1 to human analgesia is dependent upon the plasma concentrations of each compound.

Tramadol has been shown to inhibit reuptake of norepinephrine and serotonin in vitro, as have some other opioid analgesics. These mechanisms may contribute independently to the overall analgesic profile of tramadol.

Tramadol is usually marketed as the hydrochloride salt (tramadol hydrochloride) and is available in both injectable (intravenous and/or intramuscular) and oral preparations. It is also available in conjunction with paracetamol (acetaminophen).

Tramadol is approximately 10% as potent as morphine, when given by the IV/IM route. Oral doses range from 50-600 mg daily, with up to 400 mg daily when given IV/IM. The formulation containing APAP contains 37.5 mg of tramadol and 325 mg of paracetamol, intended for oral administration with a common dosing recommendation of one or two tabs every four to six hours.

As for the major formulation of orally administrable pharmaceuticals, there are solid preparations such as granules, powders or fine granules, tablets, and capsules, and liquid preparations such as syrup. Among these preparations, granules, powders or fine granules frequently give an unpleasant feeling to a person receiving such preparations, for example, a sandy feel or lodging between the teeth. Tablets or capsules are very easy to handle for recipients in comparison with granules, powders or fine granules, and widely employed as oral pharmaceutical preparations. However, these preparations are difficult to be taken by the aged or infants whose swallowing ability is weak, because those preparations sometimes stuck halfway on their esophagus. Such solid preparations are prepared in order that they are disintegrated and dissolved in the digestive organ via oral administration, and the pharmacologically active ingredient is absorbed therein; therefore, they have usually no rapid disintegration property or solubility in the oral cavity. On the other hand, syrup is an easily administrable preparation for the aged and infants, but it has a problem that the accurate measurement is hardly expected.

In this situation in pharmaceutical preparations, it has been desired to develop an intra-orally rapidly disintegrable solid preparation that can easily be taken by patients including aged persons and infants whose swallowing ability is weak, anytime and anywhere with ease without water, in view of increase of the aging population and change of the living environment, with keeping the handiness, which is a convenient characteristic of tablets. Tablets that are rapidly disintegrated in the oral cavity can easily be taken by a patient whose swallowing ability is weak at an accurate dose, different from liquid preparations such as syrup. Moreover, for other patients than the aged or infants, the tablets can be taken without water in the outdoors where no water is available, and thus, the intra-orally rapidly disintegrable solid preparation will improves the medication compliance for all of the patients.

Palatability and “mouth feel” are among the most important characteristics to be considered in providing fast dissolving or disintegrating solid dosage forms, for a drug. Unfortunately, many drugs have a bitter or otherwise unpalatable taste, or an unacceptable mouth feel, which make such drugs unsuitable for administration as fast dissolving or fast disintegrating dosage forms. Much research has been devoted to designing techniques and approaches to mask the bitter taste of drug in dosage forms. Simple approaches include adding chemicals mediating, flavoring or sweetening ingredients to the composition, which thereby mask the bitterness of the drug. When simple approaches are ineffective, drug modifying approaches are used in which the dosage form is so formulated that the drug's dissolution in the mouth is retarded or prevented by physical and/or chemical means. One such approach to retard by physical means is to embed or encapsulate the drug within a wall or barrier material that physically separates it from the saliva. Cationic copolymers synthesized from dimethylaminoethyl methacrylate and neutral methacrylic acid have been employed as the barrier material in various taste-masked formulations. In some cases, these polymers are also known to modify taste by chemically interacting with drugs.

US2006/0039981 provided a taste-masked pharmaceutical dosage faun that includes one or more drugs and one or more cationic polymers synthesized from dimethylaminoethyl methacrylate and neutral methacrylic acid esters. The wt/wt ratio of the drug to polymer is less than about one to two.

A preparation which is merely rapidly disintegrable can be easily prepared by pressing and molding the pharmaceutical components at low pressure, however, such preparation shows poor pharmaceutical strength, cannot keep their shape, and sometimes results in disintegration in the course of packaging or distribution as well as during taking-out of the preparation from the package by a recipient who intends to take it. Therefore, an intra-orally rapidly disintegrable preparations should have not only an excellent intra-oral disintegrability but also the pharmaceutical strength with no problem in handling.

Different types of dosage forms disintegrating/dissolving rapidly in the buccal cavity have been developed and dosage forms comprising a number of actives are commercially available to enhance patient compliance. For instance, in JP-B-62-50445, solid dosage forms which can be produced from aqueous solutions containing gelatin and an active ingredient by freeze drying are disclosed. And in WO93/12769, solid medicines which can be produced by drying suspension including agar are also disclosed. However, the medicines produced by the above-mentioned prior methods do not have enough hardness for packaging in bottles or blisters for storage, transport and commercial distribution. Hence, they require special pharmaceutical techniques and hence, require huge investments in plants and equipment.

EP 0,553,777 A2 and U.S. Pat. No. 5,720,974 disclose production methods for fast disintegrating tablets, wherein tablets composed of a saccharide are produced in such a manner that a saccharide mixture supplied with appropriate water is compressed at a low pressure and then dried to make solid tablets. However, such methods also require a special pharmaceutical technique and have the fear that powder composing the tablets may be adhered to the surface of a metal mold in compression process under moistening condition.

U.S. Pat. No. 5,576,014 have classified sugar-based excipients into two types on the basis of their moldability and dissolution rate. The moldability of a low molding sugar such as mannitol is improved by granulating with a high molding excipient and this is the basis of WOWTAB technology. Ethypharma has introduced an orally dispersible Flash Dose technology, which contains coated crystals and micro-granules along with disintegrants, a soluble diluent, a lubricant and optionally a swelling agent (EP 1156786 and WO 2002085336 A1)

U.S. Pat. No. 6,139,865 discloses a method of taste-masking a bitter drug by coacervation in a cyclohexane solution containing ethylcellulose and polyethylene as a phase inducer. The microcapsules thus produced are successfully incorporated in effervescent tablet formulations exhibiting acceptable taste-masking, aftertaste, and overall acceptance characteristics. This relates to use of undesirable solvents such as cyclohexane and complicated coacervation techniques which require control on particle size of microcapsules through critical process variables.

U.S. Pat. Nos. 4,305,502, 4,371,516, and 5,738,875 assigned to R. P. Scherer discloses Zydis technology based on freeze-drying, which is used to manufacture rapidly dissolving tablets. The potential drug should have a particle size <50 μm and should not have a bitter taste. Although the process is quite popular in the pharmaceutical industry, it is expensive and time-consuming. The products produced by this technology are usually fragile and require special packaging and handling.

Freeze drying processes have been used to prepare fast disintegrating dosage forms. Depending on the manufacturing process, the product obtained is characterised by a highly porous microstructure of the soluble supporting agent (i.e. mannitol, glycine, lactose, gelatins etc.) in which the active is homogeneously dispersed. Although this technology produces a product which rapidly disintegrates in water or in the oral cavity, a drawback is represented by the poor physical integrity of its physical structure which severely limits further manufacturing operations such as forming blister packs.

Another significant drawback of the freeze drying technology in manufacturing such dosage forms is the high production costs because of the lengthy duration of each freeze drying cycle (normally from 24 to 48 hours). The complexity of the industrial plants is another important factor which prejudices the large scale use of this technology for the development of rapid disintegrating tablets. Moreover, the thermal shocks, as a direct consequence of each freeze drying cycle, might physically modify the physical-chemical properties of the outer membrane of microencapsulated particles.

U.S. Pat. Nos. 5,178,878, 6,269,615 and 6,221,392, all assigned to Cima Labs, Inc., teach the art of manufacturing friable orally disintegrating tablets by direct compression and packaging in specially designed dome-shaped blister package using a robot-controlled integrated tableting-packaging system. U.S. Pat. Nos. 5,464,632 and 6,106,861 disclose methods of producing rapidly disintegrating multiparticulate tablets which disintegrate in the mouth within 60 seconds comprising an active in the form of microcrystals, coated microgranules or uncoated microgranules, 3 to 15% disintegrant, 40 to 90% of a soluble diluent which is a mixture of directly compressible polyols with an average particle size of 100 to 500μ and powdered polyols with an average particle size of less than 100μ.

U.S. Pat. No. 6,106,861 discloses a rapidly disintegratable multi-particulate tablet which disintegrates in the mouth in less than forty seconds and which comprises an excipient and an active ingredient in the form of microcrystals coated with a coating agent. The excipient comprises, with respect to the mass of the tablet, from 3 to 15% by weight of at least one disintegration agent and from 40 to 90% by weight of at least one soluble diluent agent with binding properties consisting of a polyol having less than thirteen carbon atoms, said polyol being either in the directly compressible form which is composed of particles whose average diameter is from 100 to 500 micrometers or in the powder form which is composed of particles whose average diameter is less than 100 micrometers, said polyol being selected from the group consisting of mannitol, xylitol, sorbitol and maltitol, with the proviso that, when only one soluble diluent agent with binding properties is used, it is a polyol in the directly compressible form except sorbitol and, when at least two soluble diluent agents with binding properties are used, one is consisting of a polyol in the directly compressible form and the other is consisting of the same or another polyol in powder form, the proportion of directly compressible polyol to powder polyol being from 99/1 to 50/50.

U.S. Pat. No. 6,316,029, which describes a tablet prepared by compressing a blend of ingredients into a tablet. However, the process of forming the tablet described therein may require relatively high compression forces (e.g., from 700 lbs. to 3800 lbs.). The high compression forces can result in a reduction in tablet porosity due to collapse of the void spaces during compression, thereby sacrificing the disintegration properties of the tablet.

U.S. Pat. No. 6,514,492 relates to formulation of oral liquid products of quinolones or derivatives thereof using ion exchange resins, such as methacrylic acid polymer crosslinked with divinylbenzene, as the carrier, thereby eliminating the extreme bitterness of the quinolones oral liquid formulation.

US20040253314 relates to a process for the production of taste-isolated, active ingredient-containing granules or powders by melting a mixture of a (meth)acrylate copolymer having anionic groups and of a pharmaceutical active ingredient, extrusion of the mixture comminution of the extrudate to give granules or powder. This invention includes melt granulation which may require specific equipment e.g. jacketed mixers and extruders or there may be process related problems such as milling of the solidified waxes, causing choking of equipments or heating up of milling equipment thereby again causing it to melt in the milling equipments, making the process messy and not production-friendly.

US20050042281 relates to a chewing gum composition comprising: at least one therapeutic agent at least partly in an ionized form, the ionized form capable of being converted into an un-ionized form; a gum base; a protecting agent, wherein the protecting agent coats at least a portion of the therapeutic agent and reduces adhesion between the therapeutic agent and the gum base; and a buffer system, wherein the buffer system comprises at least two different buffering agents and is capable of changing the pH of saliva from an arbitrary initial pH to a predetermined final pH, independent of the arbitrary initial pH, and of sustaining the predetermined final pH for a period of time, and wherein the buffer system favors substantially complete conversion of the ionized form to the un-ionized form. It also claims tramadol as one of the therapeutic agent that can be used in this form. However chewing gum formulations are not a good option for geriatric patients and also as the therapeutic use is analgesic, because patients needing this type of medication are already under intense pain, it will be adding more to their discomfort making them chew the gum to initiate the therapeutic action.

WO2008000453 relates to process for preparing a solid dosage form, comprising: —preparing an aqueous slurry, solution or suspension of (a) a powder material, and (b) a mixture of one or more polyols and one or more maltodextrins, and spray drying the resultant aqueous slurry, solution or suspension, thereby obtaining particles which are directly compressible into a solid dosage form being able to disintegrate in an aqueous medium within no more than 15 minutes. This invention relates to utilizing a costly equipment and process like spray-drying which may also have scale-up issues like formation of sticky mass if all process parameters are not controlled.

Additionally it may be difficult to compress the spray dried material if it has tendency to form a hygroscopic mixture. Further it might not be able to mask high dose bitter drugs like tramadol.

US20080014262 relates to a manufacturing method of a quickly disintegrating tablet which is manufactured by mixing a medicine with a saccharide, kneading the mixture with water including polyvinyl alcohol dissolved therein or an aqueous organic solvent, and subjecting to a compression-molding. These techniques often lead to noticeable decomposition of the active agent and/or the polymer, or are slow and cumbersome to use. The factors mainly responsible for their degradative effects are long heating times combined with mechanical stress caused by screws or other mixing devices in the machinery. Also it depends upon various factors such as selection of proper amount of the materials, determination of minimum time required to heat the compound as well as the appropriate heating techniques, the prediction of required force to make sure that every volume of the melt has the desired shape and the temperature of the molds should decrease rapidly during the cooling process.

US20080063710 relates to a method of such rapidly disintegrating tablet includes: (1) mixing an active ingredient, an acrylic copolymer and at least a pharmaceutically acceptable additive to obtain a mixture thereof (2) tableting the mixture to obtain a compact, and (3) isothermally heating the compact at a temperature of 50.degree. C. to 100.degree. C. for a given period of time. The process described in this application requires use of specialized equipments with thermal capacity.

There is currently an increasing demand for orally administrable formulations of pharmaceuticals, because of good associated patient compliance. However, conventional solid tablet formulations which are swallowed whole are often not ideal for administration of active ingredients. Many patients, especially the very young or old, find it difficult to swallow tablets whole.

Various preparations have been known as quickly disintegrating tablets, and particularly an intra-oral quickly disintegrating tablet which is quickly disintegrated in an oral cavity after taken has been noted in recent years. Since the intra-oral quickly disintegrating tablet can be easily taken without water, they are recently receiving public attention as a dosage form which is suitable for the people having insufficient swallowing functions such as aged people and small children.

Fast dispersing solid dosage forms for oral administration are known. The dosage forms are particularly useful for patients who have difficulty in swallowing tablets e.g. children and elderly people. In the pharmaceutical field, there is a great need for said dosage forms because many people are unwilling and/or unable to swallow tablets, capsules and other traditional solid dosage forms.

Therefore, there have been much waited for developments: of a physicochemically stable quickly disintegrating tablet including a medicine, that is, a stable oral drug product which is easy for aged people to take, whose taste or feeling during taking is good, which can be taken even by an adult having a swallowing capability without water, and of a method for producing the tablet. Concretely, for example, there have been waited for: a quickly disintegrating tablet which has a quick disintegrability and solubility in an oral cavity, has no uncomfortable tastes such as bitterness, has small variations of tablet weight, tablet hardness, tablet diameter and tablet thickness even in storage under a humidifying condition, has substantially no change of a medicine content in the tablet and tablet appearance, and is superior in stability; and for a method for producing the tablet.

The present invention provides a solid pharmaceutical dosage form adapted for direct oral administration, i.e. for direct insertion into the mouth of a patient. This is particularly useful in administration of medicaments to e.g. children, debilitated patients, patients who have difficulty swallowing solids and the elderly. Orally disintegrating tablets also are convenient under circumstances in which taking an oral dosage form with water may be inconvenient (e.g., while working or traveling).

The intra-orally rapidly disintegrable tablets of the present invention can be taken without water as mentioned above, and accordingly are particularly preferable in the following cases: (i) in the case in which administration is required frequently without water; (ii) in the case that the recipient is a patient who is difficult to swallow the tablet; and (iii) in the case that the recipient is an aged person or infant who tends to choke over the tablets of an old type.

Currently the main technologies to obtain such type of dosage forms are: (1) The active ingredient is mixed with water-soluble diluents and compressed on a tableting machine at low to medium compression force. (2) A suspension is prepared from the active ingredient and appropriate excipients, which suspension is then dispensed into blister packs and finally dried, for example freeze-dried (e.g. Zydis®). All these technologies have their drawbacks: For example in the case of (1), the mechanical resistance of the dosage forms is often insufficient in normal blister packs and the dosage forms often do not disintegrate rapidly enough (time needed for dissolution may be up to 60 seconds and more). In the case of (2), again lacking mechanical resistance in normal blister packs can be a problem but in particular the time-consuming and costly freeze-drying process is a major disadvantage. Moreover, the effectiveness of a freeze-drying process always depends on the physico-chemical parameters of the active substances used. For certain active substances, especially such having a high solubility in water, it is therefore difficult or impossible to apply a freeze-drying process and consequently this technology.

Conventional rapidly disintegrating tablets are typically formed by compression (e.g., in a tablet press). It is desirable for such tablets to have sufficiently high hardness and sufficiently low friability to provide structural stability for transportation and storage. Low friability (which is measured based on the percent tablet weight loss after a certain number of revolutions in a friabilator) is desirable in that it is generally indicative of high tablet strength. High porosity of the tablet structure also is desirable in that it allows fluids (e.g., aqueous or bodily fluids, e.g., water or saliva) to be drawn or “wicked” from the external environment and into the interstices of the tablet structure, thereby promoting rapid and effective disintegration.

Tramadol is a well-known drug disclosed in U.S. Pat. No. 3,652,589, which is used in the form of its hydrochloride salt as a non-narcotic analgesic drug. Tramadol is the pharmacologically active trans isomer of 2-dimethylaminomethyl-1-(3-methoxyphenyl)cyclohexanol, as opposed to the corresponding cis isomer, namely, (RS, SR)-2-dimethylaminomethyl-1-(3-methoxyphenyl)cyclohexanol.

Various processes for the synthesis of tramadol hydrochloride have been described in the prior art. For example, U.S. Pat. No. 3,652,589 and British patent No. 992 399 describe the preparation of tramadol hydrochloride.

U.S. Pat. No. 5,874,620 describes a process for the separation of tramadol hydrochloride from a mixture with its cis isomer, using an electrophilic reagent.

U.S. Pat. No. 6,723,343 relates to preparation of new tramadol salts with sugar substitutes to have less bitter salts of tramadol. However to use newer salts which are less bitter would require proving its efficacy and toxicity to be fit for human consumption.

Despite the excellent efficacy of tramadol hydrochloride in pain control, the active substance tramadol and its readily soluble salts have an intensely bitter taste. Immediate release conventional formulations of tramadol that releases this active substance as soon as they are taken all have this strong bitter taste. Very good water solubility of tramadol hydrochloride places additional disadvantage, as it adds to the immediate bitterness taste on the tongue when placed in the mouth to be swallowed. As a consequence, these immediate-release formulations are poorly accepted and patients fail to observe the dosage instructions. Although coating and complex processes, e.g. the application of film coatings, serve to improve taste, they may impede the immediate release of the active substance.

The processes used for taste masking in the patents listed above involve multiple steps which are technically complicated and difficult to reproduce, besides being economically disadvantageous. A major requirement of any such solid form is that it must be palatable to reduce the risk of a patient deliberately missing the medication.

Use of ion-exchange resins to form a drug-ion exchange resin complex is well known and is described, for example, in U.S. Pat. No. 2,990,332, which describes use of an ion-exchange resin to form a complex with ionic drugs and thereby delay the drug release from such complexes. However the use of ion-exchange resins are not much explored for taste-masking of bitter drugs, especially with a high dose drug such as Tramadol hydrochloride.

As such, there exists a need for a conventional rapidly disintegrating tablet having sufficiently low friability and sufficiently high hardness, while maintaining high porosity of the tablet structure. The invention provides such a tablet.

In light of the problems described for formulating an rapidly disintegrating oral composition of bitter tasting active ingredients like Tramadol and its pharmaceutically acceptable salts or solvates, the object of the present invention is to provide taste-masked rapidly disintegrating Tramadol composition which disintegrate in the oral cavity with or without the need of water for administrating such dosage forms.

The term “rapidly disintegrating tablet” in the present invention shall mean a tablet that disintegrates with a small amount of water after it is administrated or swallowed. Particularly, the term “Rapidly disintegrating tablet in the buccal cavity” in the present invention shall mean a tablet that can completely disintegrate in the oral cavity within two minutes only by saliva without water.

This invention relates to fast disintegrating tablets and in particular to Tramadol tablets which will disintegrate in the oral cavity within sixty seconds, preferably within thirty seconds.

### OBJECTS OF THE INVENTION

It is an object of the present invention to provide a rapidly disintegrating oral pharmaceutical composition of Tramadol or its pharmaceutically acceptable salts or solvates with satisfactory dissolution.

It is another object of the present invention to provide a stable, rapidly disintegrating oral pharmaceutical composition of Tramadol or its pharmaceutically acceptable salts or solvates with satisfactory dissolution.

It is another object of the present invention to provide composition for Tramadol in the form of rapidly disintegrating oral compositions, which disintegrate in the buccal cavity and their manufacturing processes. Oral composition may be in the form of tablets or a granulate.

It is another object of the present invention to provide an oral dosage form as a rapidly disintegrating tablet, which contain Tramadol as active pharmaceutical ingredient with palatable taste and mouth-feel while disintegrating in the buccal cavity and it also facilitates dissolution of the drug when in contact with the gastrointestinal fluid.

Yet another object of the present invention to provide formulation of orally disintegrating solid oral preparations of Tramadol or its pharmaceutically acceptable salts or solvates thereof using ion exchange resins, such as a methacrylic acid polymer crosslinked with divinylbenzene, as the carrier. The formation of a drug-resin complex (resinate) eliminates the extreme bitterness of the Tramadol to make the oral dosage form palatable.

### BRIEF DESCRIPTION OF THE ACCOMPANYING DRAWINGS

FIG. 1 is a graph depicting the comparative dissolution profiles of Ulram versus Tramadol.

### DETAILED DESCRIPTION OF THE INVENTION

The present invention provides a oral solid pharmaceutical composition comprising preferably at least one pharmaceutically active ingredient which has an unpleasant or bitter taste, particularly an analgesic active ingredient, most preferably the active ingredient is Tramadol or its pharmaceutically acceptable salt or solvate, said compositions being intended primarily for pediatric use, or for use in patients who find it difficult to swallow whole tablets or capsules. More particularly, the oral solid pharmaceutical composition is a solid dosage formulation in the form of rapidly disintegrating oral tablets, which disintegrate in the buccal cavity.

The rapidly disintegrating formulations comprises the active ingredient complexed with an ion-exchange resin. Drug resinates are insoluble in saliva, hence even resinate of bitter drugs have virtually no taste. With the correct selection of the ion exchange resin, the drug is not released in the mouth so that the patient does not taste the drug when it is swallowed. When the drug resinate comes into contact with the gastrointestinal fluids, such as the acid of the stomach, the drug is released from the resinate, directly into solution and then absorbed in the usual way. The resin passes through the GI tract without being absorbed.

The term “pharmaceutical composition”, as used herein, means a combination comprised of a safe and effective amount of the active ingredient, or mixtures thereof, and pharmaceutically-acceptable excipients.

The term “pharmaceutically acceptable excipients”, as used herein, means any physiologically inert, pharmacologically inactive material known to one skilled in the art, which is compatible with the physical and chemical characteristics of the particular active ingredient selected for use. Pharmaceutically-acceptable excipients include, but are not limited to, polymers, resins, plasticizers, fillers, binders, lubricants, glidants, disintegrants, solvents, co-solvents, buffer systems, surfactants, preservatives, sweetening agents, flavoring agents, pharmaceutical grade dyes or pigments, and viscosity agents.

The term “ion exchange resin”, as used herein, means anionic or cationic ion exchange resins. More preferably the ion exchange resin is composed of Methacrylic acid and Divinylbenzene polymer.

The term “oral dosage form”, as used herein, means any pharmaceutical composition intended to be systemically administered to an individual by delivering said composition to the gastrointestinal tract of an individual, via the mouth of said individual. Oral dosage forms includes orally disintegrating tablets, coated or non-coated. All percentages are on a weight percent basis unless otherwise indicated.

The term “rapidly disintegrating tablet” in the present invention shall mean a tablet that disintegrates with a small amount of water after it is administrated or swallowed. Particularly, the term “Rapidly disintegrating tablet in the buccal cavity” in the present invention shall mean a tablet that can completely disintegrate in the oral cavity within 2 minutes only by saliva without water.

The term “base granules” in the present invention is used to refer to granules devoid of the drug.

Preferably, the composition of the invention comprises at least one active ingredient which has an unpleasant and/or bitter taste. Examples of bitter or unpleasant tasting drugs include, but are not limited to, e.g. antibiotics; including macrolides, such as erythromycin or clarithromycin, penicillin, ampicillin, among others, as well as other active ingredients such as e.g. acetaminophen, caffeine, dextromethorphan, cimetidine, pseudoephedrine, diphenhydramine, zopiclone, spironolactone, chlorpheniramine, theophylline, phenylbutazone, ondansetron, cetrizine, resperidone, analgesics such as Tramadol among others.

Preferably, said active ingredients are analgesic drugs, most preferably the analgesic drug is Tramadol.

For the purpose of this invention, tramadol includes all its pharmaceutically acceptable salts, solvates, polymorphs, enantiomers or mixtures thereof. First aspect of the present invention relates to stable taste masked orally disintegrating pharmaceutical composition of tramadol comprising:
a. 3% to 30% by weight of a Tramadol or its pharmaceutically acceptable salt or solvate thereof;
b. 2% to 30% by weight of an ion exchange resin;
c. pharmaceutically acceptable excipients to equal 100%.

In one embodiment according to this aspect, the ion exchange resin used is polacrillin potassium.

In another embodiment of this aspect, the taste masked orally disintegrating pharmaceutical composition of tramadol comprises 12.82% w/w of tramadol; 10.25% w/w of polacrillin potassium resin and other pharmaceutically acceptable excipients to equal 100% w/w.

In yet another embodiment, the pharmaceutically acceptable excipients comprises of disintegrant, lubricant, filler, glidant, sweetener and flavour. More preferably, the binder used is gelatin and the disintegrant used is selected from sodium starch glycolate, crospovidone or croscarmellose sodium, the filler used is microcrystalline cellulose, lubricant used is colloidal silicon dioxide, talc, magnesium stearate, sweetener used is sucralose, sodium saccharin, flavour used is peppermint.

Second aspect of the present invention relates to stable taste masked orally disintegrating pharmaceutical composition of tramadol having fast dissolution, wherein the dissolution in 500 ml, 0.1 N HCl; (37° C.±0.5° C.), Apparatus: USP Type II (Paddle) RPM50 is not less than 70% in 5 min and not less than 85% in 10 minutes and wherein the composition comprises greater than 15% w/w of one or more of disintegrant.

In one embodiment of this aspect, the disintegrant is selected from sodium starch glycolate, crospovidone or croscarmellose sodium. Preferably, the disintegrant is used in the amount of 10 to 50% w/w. More preferably, the disintegrant used is in the amount of about 23% w/w of the composition.

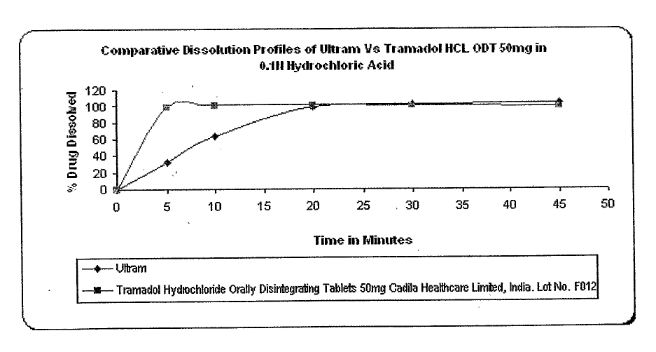

In another embodiment, the dissolution profile of the formulation of the present invention, prepared in accordance with example 1 is provided. TABLE 1 and FIG. 1 present a comparative dissolution profile of the tramadol tablets of the present invention and that of Ultram®.

Third aspect of the present invention relates to stable taste masked orally disintegrating pharmaceutical composition of tramadol, wherein such composition contains less than 0.5% w/w of total impurities, when packed in PVC/PVDC blister pack and subjected to 40±2° C./75±5% RH for 3 months.

In one embodiment of this aspect, the stability profile of the formulation of the present invention, prepared in accordance with example 1 is provided. (See TABLE 2).

Fourth aspect of the present invention relates to stable taste masked orally disintegrating pharmaceutical composition of tramadol, comprising tramadol, resin and binder, wherein the binder used is gelatin in the amount of about 0.01 to about 2% w/w of the composition.

In one embodiment of this aspect, 0.13% w/w of gelatin is used as the binder in the composition.

Fifth aspect of the present invention discloses the processes for preparation of stable taste masked orally disintegrating pharmaceutical composition of tramadol, comprising of the following steps:
(i) preparation of drug resin slurry.
(ii) preparation of binder solution.
(iii) preparation of blend comprising of filler, disintegrant and glidant.
iv. granulation of blend of step iii with binder solution of step ii to make base granules and sizing them and subsequently granulating the sized base granules with drug resin slurry of step i to prepare drug containing granules or granulation of blend of step iii with drug resin slurry followed by binder solution to prepare drug containing granules or mixing of drug resin slurry of step i with binder solution of step ii to get a homogenous mixture and granulation of blend of step iii with this homogenous mixture to prepare containing granules or
   granulation of blend of step iii with binder solution of step ii to make base granules and sizing them. Dividing the sized base granules in to two potions: large portion (80 to 99%) and small portion (20 to 1%). Keeping aside the small portion to add it during step vi along with other pharmaceutically acceptable excipients. Adding drug resin slurry to the big portion to prepare drug containing granules.
v. sizing of the drug containing granules of step iv.
vi. mixing of the sized drug containing granules with other pharmaceutically acceptable excipients like disintegrant, lubricant, glidant, sweetener and flavour or blending the sized drug containing granules with other pharmaceutically acceptable excipients like disintegrant, lubricant, glidant, sweetener, flavour and the small portion of base granules kept aside in step iv.
vii. compressing the blend of step v in to tablets.

In one embodiment of this aspect, the base granules were prepared using filler such as Microcrystalline cellulose, Sodium starch glycolate or Crospovidone or Croscarmellose sodium as disintegrant and Colloidal silicondioxide. The excipients optionally with any of the other excipients which aid the process were mixed in suitable mixer. The binder solution was prepared using binder such as gelatin in water. The excipient mixer was granulated with the gelatin binder solution to obtain a homogenous mass. The wet mass was dried in a suitable drier such as an FBD to achieve moisture content below 2.5% w/w. The dried granules were sifted on a vibratory sifter and over-sized granules were milled using a suitable mill to get an optimum sized granules. These are called base granules.

The ion exchange resin was added in water in a stainless steel tank equipped with a suitable stirrer. The active ingredient, Tramadol hydrochloride was added to the resin slurry under continuous stirring and stirred further for at least 30 mins and up to 180 mins. The base granules were charged into a rapid mixer granulator and granulated with activated resin-Active slurry. The wet mass was dried in FBD to achieve moisture content below 3% w/w. The dried granules were sifted on a vibratory sifter and over-sized granules were milled using a suitable mill to get an optimum sized granules. The sized granules were mixed with previously sifted excipients such as disintegrants, glidants, lubricants, flavours, sweeteners in a suitable blender for 10 mins. The lubricated blend was compressed into tablets using suitable tablet toolings at desired hardness with a hardness range from 2 to 8 Kp, an average of about 3 Kp hardness and friability less than 0.5% w/w for 500 revolutions per minute to achieve a disintegrating time of less than 60 secs and more preferably around or less than 30 secs.

In another embodiment, Microcrystalline cellulose, Sodium starch glycolate or Crospovidone or Croscarmellose sodium as disintegrant and Colloidal silicondioxide were mixed in a rapid mixer granulator to get a uniform blend. The ion exchange resin was added in water in a stainless steel vessel equipped with a suitable stirrer. The active ingredient, Tramadol hydrochloride was added to the resin slurry under continuous stirring and stirred further for at least 30 mins and up to 180 mins. Poured resin-active slurry over the dry mix with slow addition and mixing. Gelatin solution was prepared in water. The mass was further granulated with the gelatin solution. The wet mass was dried in FBD to achieve moisture content below 3% w/w. The dried granules were sifted on a vibratory sifter and over-sized granules were milled using a suitable mill to get an optimum sized granules. The sized granules were mixed with previously sifted excipients such as disintegrants, glidants, lubricants, flavours, sweeteners in a suitable blender for 10 mins. The lubricated blend was compressed into tablets using suitable tablet toolings at desired hardness with a hardness range from 2 to 8 Kp, an average of about 3 Kp hardness and friability less than 0.5% w/w for 500 revolutions per minute to achieve a disintegrating time of less than 60 secs and more preferably around or less than 30 secs.

In yet another embodiment, Microcrystalline cellulose, Sodium starch glycolate or Crospovidone or Croscarmellose sodium as disintegrant and Colloidal silicondioxide were mixed in a rapid mixer granulator to get a uniform blend. The ion exchange resin was added in water in a stainless steel vessel equipped with a suitable stirrer. The active ingredient, Tramadol hydrochloride was added to the resin slurry under continuous stirring and stirred further for at least 30 mins and up to 180 mins. Gelatin solution was prepared in water. Add Active-resin slurry into gelatin solution under continuous stirring to obtain a homogenous mass. The dry mix was granulated with the gelatin-active-resin solution with slow addition and slow kneading to obtain a homogenous granulated wet mass. The wet mass was dried in FBD to achieve moisture content below 3% w/w. The dried granules were sifted on a vibratory sifter and over-sized granules were milled using a suitable mill to get an optimum sized granules. The sized granules were mixed with previously sifted excipients such as disintegrants, glidants, lubricants, flavours, sweeteners in a suitable blender for 15 mins. The lubricated blend was compressed into tablets using suitable tablet toolings at desired hardness with a hardness range from 2 to 8 Kp, an average of about 3 Kp hardness and friability less than 0.5% w/w for 500 revolutions per minute to achieve a disintegrating time of less than 60 secs and more preferably around or less than 30 secs.

In yet another embodiment, Microcrystalline cellulose, Sodium starch glycolate or Crospovidone or Croscarmellose sodium as disintegrant and Colloidal silicondioxide were mixed in a rapid mixer granulator to get a uniform blend. Gelatin solution was prepared in water. The dry mix was granulated with the gelatin solution with slow addition and slow kneading to obtain a homogenous granulated wet mass. The wet mass was dried in FBD to achieve moisture content below 3% w/w. The dried granules were sifted on a vibratory sifter and over-sized granules were milled using a suitable mill to get an optimum sized granules. This forms the base granules. The base granules were divided into two parts, approximately 80-99% of the total base granules was charged in the rapid mixer granulator. The ion exchange resin was added in water in a stainless steel vessel equipped with a suitable stirrer. The active ingredient, Tramadol hydrochloride was added to the resin slurry under continuous stirring and stirred further for at least 30 mins and up to 180 mins. The base granules in rapid mixer granulator was granulated with the active-resin slurry with slow addition and slow kneading to obtain a homogenous granulated wet mass. The wet mass was dried in FBD to achieve moisture content below 3.5% w/w. The dried granules were sifted on a vibratory sifter and over-sized granules were milled using a suitable mill to get an optimum sized granules. The sized granules were mixed with previously sifted excipients such as disintegrants, glidants, lubricants, flavours, sweeteners along with 1-20% of remaining base granules in a suitable blender for 15 mins. The lubricated blend was compressed into tablets using suitable tablet toolings at desired hardness with a hardness range from 2 to 8 Kp, an average of about 3 Kp hardness and friability less than 0.5% w/w for 500 revolutions per minute to achieve a disintegrating time of less than 60 secs and more preferably around or less than 30 secs.

In any of the above embodiments, the granules can be granulated with or without use of binder solution of gelatin or any other suitable binder known to a person skilled in the art.

In any of the above embodiments, the sequence of granulation with addition of active-resin slurry and binder solution can be varied to achieve desired tablet characteristics.

In any of the above embodiments the % age of binder solution containing gelatin or any other suitable binder can range between 0% to 5% w/w of the formulation. More preferably the binder concentration will be below 3% w/w of the formulation. More preferably the binder concentration will be below 1% w/w of the formulation.

In any of the above embodiments the % age of flavours or sweeteners can range between 0.05% to 2% w/w of the formulation.

The tablets of the present invention were subjected to in-process analysis, wherein in-process tests like description, identification, thickness, hardness, friability, disintegration time and uniformity of dosage units were performed. (See TABLE 3). The in process test results showed that the in process variables were well within the limits of the acceptance criteria.

Ion exchange resins useful in the present invention include, but are not limited to, anionic resins such as: DUOLITE® AP143/1083 (cholestyramine resin USP) and cationic resins such as: AMBERLITE® IRP-64 (a porous copolymers of methacrylic acid crosslinked with divinylbenzene), AMBERLITE® IRP-69 (Sodium polystyrene sulfonate USP) and AMBERLITE® IRP-88 (Polacrilin Potassium). AMBERLITE® IRP 88 is preferred resin. The DUOLITE® and AMBERLITE® resins are available from the Rohm and Haas Company, Philadelphia, Pa. The DOWEX® resins, available from the Dow Chemical Company, Midland, Mich. are also useful in the practice of the present invention. Said DOWEX® resins are strong cationic exchangers based upon polystyrenesulphonic acid with variable crosslinking (1-12% divinylbenzene) in a variety of particle sizes.

Further, said AMBERLITE® IRP 88 (Polacrilin Potassium) is available commercially as a potassium salt. However, it is within the scope of the present invention to convert the potassium salt to other salt forms including, but not limited to, Na and Li.

Polacrillin potassium useful in the present invention can also be available as other brands such as Indion 294, sourced from Ion Exchange Resin India Ltd.

The ion exchange resins useful in the practice of the present invention comprise from about 2% to about 30% by weight of the pharmaceutical compositions of the present invention. Preferably the ion exchange resins useful in the practice of the present invention comprise from about 5% to about 20% by weight of the pharmaceutical compositions of the present invention. In one embodiment, the ion exchange resins used is about 10% w/w of the composition. The ratio of tramadol to resin in the formulation can be from about 1:0.1 to about 0.1:1.

In one of the preferred embodiment, the ratio of tramadol to resin in the formulation is 1:0.8.

As stated hereinabove, pharmaceutically-acceptable excipients include, but are not limited to, resins, fillers, binders, lubricants, glidants, disintegrants, surfactants, preservatives, sweetener agents, flavoring agents, buffer systems, pharmaceutical-grade dyes or pigments, etc.

Disintegrants which can be utilized in the formulation of the present invention include, but are not limited to, methylcellulose, cellulose, carboxymethylcellulose, sodium carboxymethylcellulose, calcium carboxymethylcellulose, croscarmellose sodium, magnesium aluminum silicate, povidone, starch, sodium starch glycolate, pregelatinized starch, hydroxy propylmethylcellulose. Preferably, the disintegrant is sodium starch glycolate, croscarmellose sodium or crospovidone.

Suitable binders for use in the present formulation and processes include, but are not limited to, synthetic gums, celluloses, hydroxypropyl methylcellulose, polyvinyl pyrrolidone (povidone), carboxymethylcellulose, ethylcellulose and methylcellulose, starch, pregelatinized starch, gelatin, sugars. More preferably, the binder is povidone or gelatin. Gelatin is sourced from Roussel. The ratio of tramadol to gelatin in the formulation can be is from about 200:0.1 to about 10:1.

In one of the preferred embodiment, the ratio of tramadol to gelatin in the formulation can be is 100:1.

Flavoring agents among those useful herein include those described in Remington's Pharmaceutical Sciences, 18th Edition, Mack Publishing Company, 1990, pp. 1288-1300, incorporated by reference herein. The pharmaceutical compositions suitable for use herein generally contain from 0-5% flavoring agents.

Preferred sweeteners include, but are not limited to, sucrose, glucose, saccharin, sorbitol, malt extract, mannitol, sucralose and aspartame. Particularly preferred is sodium saccharin and sucralose. Sweeteners such as saccharin and sucralose are generally used at levels of 0.1% to 5%.

The compositions of the present invention may optionally contain lactose, mannitol, sorbitol, tribasic calcium phosphate, dibasic calcium phosphate, compressible sugar, starch, calcium sulfate, dextro and microcrystalline cellulose, magnesium stearate, stearic acid, talc, colloidal silicon dioxide, starch, sodium starch glycolate, crospovidone, croscarmellose sodium, and microcrystalline cellulose, acacia, tragacanth, hydroxypropylcellulose, pregelatinized starch, gelatin, povidone, ethylcellulose, hydroxypropylcellulose, hydroxypropylmethylcellulose, and methylcellulose.

The process described in the present invention is demonstrated in example illustrated below. This example is provided as illustration only and therefore should not be construed as limitation of the scope of invention.

### Examples: 1, 2 and 3

| | Name | mg/tab | | | |
|---|---|---|---|---|---|
| S No | of Ingredient | Example 1 | Example 2 | Example 3 | Example 4 |
| A | Stage A Drug | | — | | |
| | Resin Slurry | | | | |
| 1. | Tramadol | 50.00 | 50.00 | 50.00 | 50.00 |
| | Hydrochloride | | | | |
| 2. | Polacrilin | 40.00 | 40.00 | 40.00 | 40.00 |
| | Potassium | | | | |
| 3. | Purified Water* | q.s | q.s | q.s | q.s |
| B | Stage B Basic | — | — | — | — |
| | Granules | | | | |
| 1. | Microcrystalline | 194.800 | 180.50 | 180.50 | 180.50 |
| | Cellulose | | | | |
| 2. | Sodium Starch | 60.000 | 60.00 | — | — |
| | Glycolate | | | | |
| 3. | Crospovidone | | — | 60.00 | — |
| 4. | Croscarmellose | | — | — | 60.00 |
| | sodium | | | | |
| 5. | Colloidal Silicon | 1.000 | 5.00 | 5.00 | 5.00 |
| | Dioxide | | | | |
| 6. | Gelatin | 0.200 | 0.50 | 0.50 | 0.50 |
| 7. | Purified Water | q.s | q.s | q.s | q.s |
| C | Stage C | | — | — | — |
| | Lubrication | | | | |
| 1. | Sucralose | 4.000 | 4.00 | 4.00 | 4.00 |
| 2. | Sodium Saccharin | 1.000 | 1.00 | 1.00 | 1.00 |
| 3. | Peppermint | 5.000 | 5.00 | 5.00 | 5.00 |
| | Flavor | | | | |
| 4. | Colloidal Silicon | 4.000 | 4.00 | 4.00 | 4.00 |
| | Dioxide | | | | |
| 5. | Sodium Starch | 30.000 | 30.00 | — | — |
| | Glycolate | | | | |
| 6. | Crospovidone | — | — | 30.00 | — |
| 7. | Croscarmellose | — | — | — | 30.00 |
| | sodium | | | | |
| 8. | Menthol | 1.000 | 1.00 | 1.00 | 1.00 |
| 9. | Talc | 7.000 | 7.00 | 7.00 | 7.00 |
| 10. | Magnesium | 2.000 | 2.00 | 2.00 | 2.00 |
| | Stearate | | | | |
| | Total | 400.00 | 390.00 | 390.00 | 390.00 |

### Process for Examples 1, 2, 3 and 4

1. Purified water was taken in a SS tank equipped with a stirrer, Polacrilin Potassium was added to water and stirred for 30 minutes to activate resin
2. Tramadol hydrochloride was added slowly under continuous stirring and stirred further for at least 30 mins.
3. Microcrystalline Cellulose, Sodium Starch Glycolate/Crospovidone/Croscarmellose sodium and Colloidal Silicon Dioxide were mixed in a rapid mixer granulator at slow speed for 5 min to obtain a uniform blend.
4. Gelatin solution was prepared in purified water.
5. Granulated blend of step 3 with gelatin solution of step 4 with slow binder addition and operating the granulator at slow speed.
6. The wet mass was dried in FBD at an inlet temperature of 85° C., to achieve a moisture content of not more than 2.5% w/w.
7. Dried granules were sifted on a vibratory sifter with 20 mesh sieve and over sized granules were milled using suitable screen to obtain 20 mesh granules.
8. The step 7 base granules were charged in RMG, and drug resin slurry of step 2 was added on base granules and granulated for 5 minutes at slow speed.
9. The wet mass was dried in FBD to achieve a moisture content of not more than 3.0% w/w.
10. Dried granules were sifted on a vibratory sifter with 20 mesh sieve and over sized granules were milled using suitable screen to obtain 20 mesh granules.
11. Sized granules were blended with pre-sifted (through 30 mesh) Stage C excipients and lubricants, in a suitable blender.
12. Lubricated blend was compressed into tablets using 10.90 mm punch sets on a rotary tablet press.

### Examples: 5 and 6

### Example 5

### Same as Example 2

### Example 6

### Same as Example 3

### Process for Example 5 and 6

1. Purified water was taken in a ss tank equipped with a stirrer, Polacrilin Potassium was added and stirred for 30 minutes to activate resin.
2. Tramadol Hydrochloride was added slowly under continuous stirring and stirred further for at least 30 mins.
3. Microcrystalline Cellulose, Sodium Starch Glycolate/Crospovidone and Colloidal Silicon Dioxide were mixed in rapid mixer granulator at slow speed for 5 min to obtain a uniform blend
4. API-Resin slurry (of step 2) was poured on powder blend (of step 3) slowly and kneaded for 3 to 5 minutes at slow speed.
5. Gelatin solution (binder) was prepared in purified water.
6. Blend of step 4 was further granulated with gelatin solution of step 5
7. Wet granules were dried in FBD to achieve a moisture content of not more than 3.0% w/w.
8. Dried granules were sifted on a vibratory sifter with 20 mesh sieve and over sized granules were milled using suitable comminuting mill to obtain 20 mesh granules.
9. Sized granules of step 8 and pre-sifted (through 30 mesh) Stage C excipients and lubricants were blended in a suitable blender for 10 minutes
10. Lubricated blend was compressed into tablets using 10.90 mm punch sets on a rotary tablet press.

### Example: 7 and 8

### Example 7

### Same as Example 2

### Example 8

### Same as Example 3

### Process for Examples 7 and 8

1. Purified water was taken in a SS tank equipped with a stirrer, Polacrilin Potassium was added to water and stirred for 30 minutes to activate resin
2. Tramadol Hydrochloride was slowly added to Step 1 under continuous stirring and stirred further for at least 30 mins.
3. Microcrystalline Cellulose, Sodium starch glycolate/Crospovidone, Colloidal Silicon Dioxide were mixed in a rapid mixer granulator at slow speed for 5 min to obtain a uniform blend.
4. Gelatin solution was prepared in water.
5. API-Resin Slurry was added into above gelatin solution under stirring to obtain a homogeneous mass.
6. Binder solution of step 5 (containing gelatin-API-resin in purified water) was added slowly over a period of 3 to 5 minutes on dry blend of step 3 and granulated.
7. Wet mass was dried in FBD to achieve a moisture content of not more than 3.0% w/w.
8. Dried granules were sifted on a vibratory sifter with 20 mesh sieve and over sized granules were milled using suitable screen to obtain 20 mesh granules.
9. Sized granules of step 8 & pre-sifted (through 30 mesh) Stage C excipients and lubricants were blended in a suitable blender for 15 minutes
10. Lubricated blend were compressed into tablets using 10.90 mm punch sets on a rotary tablet press.

### Examples: 9 and 10

### Example 9

### Same as Example 2

### Example 10

### Same as Example 3

### Process for Examples 9 and 10

1. Purified water was taken in a SS tank equipped with a stirrer, Polacrilin Potassium was added to water and stirred for 30 minutes to activate resin
2. Tramadol Hydrochloride was added slowly under continuous stirring and stirred further for at least 30 minutes.
3. Microcrystalline Cellulose, Sodium Starch Glycolate/Crospovidone and Colloidal Silicon Dioxide were mixed in a rapid mixer granulator for 5 to 15 min at slow speed to obtain a uniform blend.
4. Gelatin solution was prepared in water.
5. Dry blend of step 3 was granulated by slowly adding above binder solution of step 4 over a period of 3 to 5 minutes.
6. Wet mass was dried in FBD to achieve a moisture content of not more than 3.0% w/w.
7. Dried granules were sifted on a vibratory sifter with 20 mesh sieve and over sized granules were milled using suitable comminuting mill to obtain 20 mesh granules.
8. Base granules of step 7 were divided in two parts.
9. 99% of the base granules were charged in RMG.
10. Drug resin slurry of step 2 was slowly added on base granules and granulated for 3 to 5 minutes at slow speed.
11. Wet mass was dried in FBD to achieve a moisture content of not more than 3.5% w/w.
12. Dried granules were sifted on a vibratory sifter with 20 mesh sieve and over sized granules were sifted using suitable comminuting mill to obtain 20 mesh granules.
13. Sized granules and pre-sifted (through 30 mesh) Stage C excipients and lubricants along with remaining 1% of base granules from Step 8 were blended in a suitable blender for 15 minutes
14. Lubricated blend was compressed into tablets using 10.90 mm punch sets on a rotary tablet press.

### Examples: 11 and 12

| | | mg/tab | |
|---|---|---|---|
| S. No. | Name of Ingredient | Example 11 | Example 12 |
| A | Stage A Drug Resin | — | — |
| | Slurry | | |
| 1. | Tramadol Hydrochloride | 50.00 | 50.00 |
| 2. | Polacrilin Potassium | 100.00 | 9.98 |
| 3. | Purified Water* | q.s | q.s |
| B | Stage B Basic Granules | — | — |
| 1. | Microcrystalline Cellulose | 120.51 | 210.52 |
| 2. | Sodium Starch Glycolate | 60.00 | 60.00 |
| 3. | Colloidal Silicon Dioxide | 5.00 | 5.00 |
| 4. | Gelatin | 0.50 | 0.50 |
| 5. | Purified Water* | q.s | q.s |
| C | Stage C Lubrication | — | — |
| 1. | Sucralose | 4.00 | 4.00 |
| 2. | Sodium Saccharin | 1.00 | 1.00 |
| 3. | Peppermint Flavor | 5.00 | 5.00 |
| 4. | Colloidal Silicon Dioxide | 4.00 | 4.00 |
| 5. | Sodium Starch Glycolate | 30.00 | 30.00 |
| 6. | Menthol | 1.00 | 1.00 |
| 7. | Talc | 7.00 | 7.00 |
| 8. | Magnesium Stearate | 2.00 | 2.00 |
| | Total | 390.00 | 390.00 |

### Process for Examples 11 and 12

### Same as the Process for Examples 9 and 10

The tablets of the examples described were tested for disintegration time using USP disintegrating test apparatus with 900 ml water and 28-32 cycles/min. The tablets made by the above examples and processes showed a disintegrating time of less than 60 seconds and some of the examples even showed disintegration time less than 30 secs.

Examples 2-10 exhibited a disintegrating time of 24 secs, 18 secs, 27 secs, 30 secs, 29 secs, 42 secs, 36 secs, 20 secs and 15 secs respectively.

The tablets made by the above described examples are easy to process without requiring any complicated or costly equipments, exhibit rapid disintegration, and with pleasant mouth-feel and good organoleptic properties.

Thus the tablets prepared by using ion-exchange resins in the present invention disintegrate rapidly in the buccal cavity that can completely disintegrate in the oral cavity within 2 minutes and more preferably within 60 seconds only by saliva without water.

TABLE 1

| | | | | | | % Drug Dissolved | | | | |
|---|---|---|---|---|---|---|---|---|---|---|
| % Drug Dissolved (Ultram ®) | | | | | | (Tablets of present invention) | | | | |
| Unit | 5 | 10 | 20 | 30 | 45 | 5 | 10 | 20 | 30 | 45 |
| No. | min | min | min | min | min | min | min | min | min | min |
| 1. | 30.9 | 67.6 | 88.2 | 101.8 | 104.7 | 101.7 | 105.2 | 104.0 | 101.8 | 101.2 |
| 2. | 34.2 | 65.5 | 101.4 | 102.2 | 102.7 | 100.5 | 103.2 | 102.2 | 100.8 | 100.3 |
| 3. | 35.8 | 61.6 | 101.7 | 102.8 | 100.9 | 98.3 | 102.8 | 102.2 | 101.3 | 100.3 |
| 4. | 28.4 | 63.3 | 98.0 | 101.5 | 104.7 | 96.2 | 101.5 | 100.7 | 100.2 | 98.8 |
| 5. | 30.9 | 59.7 | 101.6 | 102.1 | 104.5 | 99.0 | 97.8 | 97.5 | 97.0 | 97.0 |
| 6. | 27.8 | 64.2 | 95.5 | 100.2 | 104.4 | 99.3 | 102.7 | 101.3 | 101.0 | 99.2 |
| 7. | 37.3 | 63.4 | 100.5 | 102.8 | 104.3 | 100.5 | 103.2 | 102.2 | 100.8 | 100.3 |
| 8. | 33.0 | 61.8 | 96.8 | 101.8 | 104.2 | 98.3 | 102.8 | 102.2 | 101.3 | 100.3 |
| 9. | 28.7 | 63.6 | 101.3 | 103.7 | 104.5 | 96.2 | 101.5 | 100.7 | 100.2 | 98.8 |
| 10. | 29.1 | 65.8 | 100.4 | 102.5 | 103.9 | 99.0 | 97.8 | 97.5 | 97.0 | 97.0 |
| 11. | 37.0 | 59.4 | 96.5 | 102.7 | 102.6 | 101.7 | 105.2 | 104.0 | 101.8 | 101.2 |
| 12. | 35.5 | 67.4 | 101.1 | 104.1 | 103.4 | 99.3 | 102.7 | 101.3 | 101.0 | 99.2 |
| Mean | 32.4 | 63.6 | 98.6 | 102.4 | 103.7 | 99.17 | 102.20 | 101.3 | 100.35 | 99.47 |
| RSD | 10.9 | 4.2 | 4.0 | 1.0 | 1.1 | 1.79 | 2.3 | 2.0 | 1.6 | 1.4 |
| Min. | 27.8 | 59.4 | 88.2 | 100.2 | 100.9 | 96.2 | 97.8 | 97.5 | 97.0 | 97.0 |
| Max. | 37.3 | 67.6 | 101.7 | 104.1 | 104.7 | 101.7 | 105.2 | 104.0 | 101.8 | 101.2 |

TABLE 2

| | Related Substances | | | Dissolution |
|---|---|---|---|---|
| | Maximum Unknown | Total | | As per OGD |
| Station | (Specify RRT*) | Impurities | Assay | Parameters |
| Initial | BQL | Nil | 105.5 | 106.4 |
| 1 Months | 0.03 (RRT-1.13) | 0.06 | 102.3 | NA |
| 2 Months | 0.06 (RRT-1.13) | 0.09 | 100.9 | NA |
| 3 Months | 0.05 (RRT-1.13) | 0.05 | 101.2 | 98.5 |

*Relative Retention Time;
NA: Not carried out.

TABLE 3

| In-Process Test | Acceptance Criteria | Results | | | |
|---|---|---|---|---|---|
| Description: | White to off white, round, | White to off white, round, uncoated | | | |
| | uncoated tablets debossed | tablets debossed with ‘320’ on one | | | |
| | with ‘320’ on one side and plain | side and plain on the other side. | | | |
| | on the other side. | | | | |
| Identification | The retention time of the | The retention time of the major peak | | | |
| (By HPLC) | major peak in the | in the chromatogram of the sample | | | |
| | chromatogram of the | preparation corresponds to that in the | | | |
| | sample preparation | chromatogram of the standard | | | |
| | corresponds to that in the | preparation, as obtained in the assay. | | | |
| | chromatogram of the | | | | |
| | standard preparation, as | | | | |
| | obtained in the assay. | | | | |
| Thickness (mm) | 5.6 ± 0.50 | 5.74 | | | |
| Hardness (kp) | 2.5-9.0 | 6.2 | | | |
| Friability(% | NMT 1.0 | 0.04 | | | |
| w/w) | | | | | |
| Disintegration | NMT 60 seconds | 20 seconds | | | |
| time (min) | | | | | |
| Uniformity of | Mean of all results is | S1 | 105.6% | S6 | 104.8% |
| Dosages units | 90.0% to 110.0% of | S2 | 105.6% | S7 | 105.7% |
| (By HPLC) | target assay. | S3 | 104.8% | S8 | 105.5% |
| (For Tramadol | | S4 | 104.7% | S9 | 104.6% |
| Hydrochloride) | | S5 | 105.0% | S10 | 104.8% |

| | |
|---|---|
| Mean | 105.1 |
| Min | 104.6 |
| Max | 105.7 |

| | |
|---|---|
| (ii) RSD of all individual | 0.4% |
| results: Not more than | |
| 5.0% | |

1. A stable taste-masked orally disintegrating pharmaceutical composition comprising:
(a) 3% to 30% w/w of a Tramadol and
(b) 2% to 30% w/w of an ion exchange resin;

2. A stable taste-masked orally disintegrating pharmaceutical composition according to claim 1, wherein the tramadol is in the form of resinate.

3. A stable taste-masked orally disintegrating pharmaceutical composition according to claim 1, wherein the ion exchange resin is polacrillin potassium.

4. A taste-masked orally disintegrating pharmaceutical composition comprising tramadol, resin and a binder, wherein the binder is gelatin.

5. A stable taste-masked orally disintegrating pharmaceutical composition according to claim 4, wherein gelatin is used in the range of about 0.01 to about 2% w/w of the composition.

6. A stable taste-masked orally disintegrating pharmaceutical composition comprising:
(a) about 13% w/w of tramadol
(b) about 10% w/w of polacrillin potassium and
(c) about 0.13% w/w of gelatin.

7. A stable taste-masked orally disintegrating pharmaceutical composition of tramadol, wherein the composition comprises greater than 15% w/w of one or more of disintegrant and having fast dissolution, releasing not less than 70% of drug in 5 min and not less than 85% of drug in 10 minutes.

8. A stable taste-masked orally disintegrating pharmaceutical composition of tramadol, according to claim 1, wherein such composition contains less than 0.5% w/w of total impurities.

9. A process of preparing stable taste-masked orally disintegrating pharmaceutical composition comprising of the following steps:
(i) preparation of drug resin slurry.
(ii) preparation of binder solution.
(iii) preparation of blend comprising of filler, disintegrant and glidant.
(iv) granulation of blend of step iii with binder solution of step ii to make base granules and sizing them and subsequently granulating the sized base granules with drug resin slurry of step i to prepare drug containing granules or
granulation of blend of step iii with drug resin slurry followed by binder solution to prepare drug containing granules or mixing of drug resin slurry of step i with binder solution of step ii to get a homogenous mixture and granulation of blend of step iii with this homogenous mixture to prepare containing granules or
granulation of blend of step iii with binder solution of step ii to make base granules and sizing them. Dividing the sized base granules in to two potions: large portion (80 to 99%) and small portion (20 to 1%). Keeping aside the small portion to add it during step vi along with other pharmaceutically acceptable excipients. Adding drug resin slurry to the big portion to prepare drug containing granules.
(v) sizing of the drug containing granules of step iv.
(vi) mixing of the sized drug containing granules with other pharmaceutically acceptable excipients like disintegrant, lubricant, glidant, sweetener and flavour or
blending the sized drug containing granules with other pharmaceutically acceptable excipients like disintegrant, lubricant, glidant, sweetener, flavour and the small portion of base granules kept aside in step iv.
(vii) compressing the blend of step v in to tablets.

10. A process for preparing stable taste-masked orally disintegrating pharmaceutical composition, according to claim 9, wherein the drug is tramadol, resin is polacrillin potassium, binder is gelatin and disintegrant is selected from the group consisting of sodium starch glycolate, crospovidone and croscarmellose.

11. A taste-masked orally disintegrating pharmaceutical composition according to claim 1, wherein the composition comprises of tramadol, polacrillin potassium resin, binder, disintegrant, filler, sweetener, flavour and a lubricant.

12. A taste-masked orally disintegrating pharmaceutical composition according to claim 1, wherein the composition comprises of:
(a) about 3 to 30% w/w of tramadol
(b) about 2 to 30% w/w of polacrillin potassium
(c) about 0.01 to 2% w/w of gelatin
(d) about 20 to 60% w/w of microcrystalline cellulose
(e) about 15 to 30% w/w of disintegrant
(f) about 0.25 to 3% w/w of glidant
(g) about 0.25 to 5% w/w of lubricant
(h) about 0.25 to 5% w/w of sweetener
(i) about 0.1 to 5% w/w of flavour.

13. A taste-masked orally disintegrating pharmaceutical composition of tramadol according to claim 1, wherein the composition comprises of greater than 15% w/w of a disintegrant, which is selected from the from the group consisting of sodium starch glycolate, crospovidone and croscarmellose.

14. A taste-masked orally disintegrating pharmaceutical composition according to claim 4, wherein the composition comprises of tramadol, polacrillin potassium resin, binder, disintegrant, filler, sweetener, flavour and a lubricant.

15. A taste-masked orally disintegrating pharmaceutical composition of tramadol according to claim 4, wherein the composition comprises of greater than 15% w/w of a disintegrant, which is selected from the from the group consisting of sodium starch glycolate, crospovidone and croscarmellose.
